(19) United States
(12) Patent Application Publication
(10) Pub. No.: US 2009/0299270 A1
(43) Pub. Date: Dec. 3, 2009

(54) LINE OF PERFUSION FOR LIQUID OF MEDICAL TREATMENT

(75) Inventor: Philippe BUISSON, Toussieu (FR)

Correspondence Address: CANTOR COLBURN, LLP, 20 Church Street, 22nd Floor, Hartford, CT, 06103, US

(73) Assignee: DORAN INTERNATIONAL, Toussieu (FR)

(21) Appl. No.: 12/173,145

(22) Filed: Jul. 15, 2008

Related U.S. Application Data
(60) Provisional application No. 61/057,095, filed on May 29, 2008.

(30) Foreign Application Priority Data
May 28, 2008 (FR) 08 02893

(51) Int. Cl. A61M 5/00; A61M 39/28

(52) U.S. Cl. 604/28; 604/34

(57) ABSTRACT

The perfusion line includes a tap having two fluid inlet ways and one fluid outlet way, each fluid inlet way being fitted with luer type connector means, one of the fluid inlet ways including stopping means that can be deformed or moved between a first position in which the stopping means prevent a flow of fluid through the fluid inlet way including the stopping means, and a second position in which the stopping means allow a flow of fluid through the fluid inlet way including the stopping means, and an extender of which one of the ends, called the proximal end, is mounted on the fluid outlet way of the tap and of which the other end, called the distal end, is fitted with means for connection to a catheter.

### CROSS REFERENCE TO RELATED APPLICATIONS

This application is related to U.S. Provisional Patent Application Ser. No. 61/057,095 filed on 29 May 2008 and further claims priority under 35 U.S.C. 119(a) to French patent application Ser. No. 08/02893 filed on 28 May 2008, which both of said applications are herein incorporated by reference in their entirety.

### TECHNICAL FIELD OF INVENTION

The present invention relates to a perfusion line for the administration of medical treatment liquids, such as chemotherapy products, to a patient, and the method for using such a perfusion line.

### BRIEF SUMMARY OF RELATED ART

A perfusion line for the administration of medical treatment liquids to a patient, such as chemotherapy products, in a known manner comprises:
a tap comprising a fluid inlet way comprising means for connection to a syringe, and a fluid outlet way, and
an extender of which one of the ends, called the proximal end, is mounted on the fluid outlet way and of which the other end, called the distal end, is fitted with means for connection to a catheter.

The method for using such a perfusion line comprises the following steps consisting in:
connecting a first flushing syringe containing a flushing product, such as physiological serum or glucose, to the fluid inlet way of the tap,
draining the tap and the extender by injecting into the latter a sufficient quantity of flushing product contained in the flushing syringe,
disconnecting the flushing syringe,
connecting an administration syringe containing a medical treatment liquid to the fluid inlet way of the tap,
injecting the medical treatment liquid as far as the catheter with the aid of the administration syringe,
disconnecting the extender of the catheter.

The use of such a perfusion line involves steps that are dangerous for hospital personnel, notably during the administration of chemotherapy products because of the handling of these products by the hospital personnel.

Specifically, during the preparation of the chemotherapy product and of the mixture of the various products forming the latter, the hospital personnel may receive splashes of the chemotherapy product or inhale vapours of the active principle of the chemotherapy product.

In addition, the hospital personnel and the patient may also receive splashes of the chemotherapy product or inhale vapours of this product during the disconnection of the catheter extender since a considerable quantity of chemotherapy product still remains inside the extender.

In addition, the presence of a considerable quantity of chemotherapy product in the extender at the end of use of the perfusion line constitutes a waste of product and involves discarding waste that is hazardous for the environment.

### BRIEF SUMMARY OF THE INVENTION

The technical problem at the base of the invention is therefore the production of a perfusion line for the administration of medical treatment liquids that has a simple and economic structure, whose use makes it possible to protect a user and the patient to be treated from any contact with the treatment liquids intended to be administered to the latter, while protecting the environment.

Accordingly, the present invention relates to a perfusion line for the administration of medical treatment liquids, such as chemotherapy products, to a patient, characterized in that it comprises:
a tap comprising at least two fluid inlet ways and a fluid outlet way, each fluid inlet way being fitted with luer type connector means arranged to allow the connection of a receptacle, such as a syringe, at least one of the fluid inlet ways comprising stopping means that can be deformed or moved between a first position in which the stopping means prevent a flow of fluid through the fluid inlet way comprising the said stopping means, and a second position in which the stopping means allow a flow of fluid through the fluid inlet way comprising the said stopping means, the stopping means being arranged to be deformed or moved into the second position when a receptacle, such as a syringe, is connected to the fluid inlet way comprising the said stopping means,
an extender of which one of the ends, called the proximal end, is mounted on the fluid outlet way of the tap and of which the other end, called the distal end, is fitted with means for connection to an element forming a catheter.

It should be noted that “chemotherapy product” means any product allowing the treatment of a cancer.

Preferably, the stopping means comprise an elastically deformable split stopper.

Advantageously, the means of connection to an element forming a catheter comprise a fitting of the male luer-lock type.

According to one embodiment of the invention, the luer type connector means of each fluid inlet way comprise a fitting of the female luer-lock type.

Preferably, the tap is a three-way tap.

The present invention also relates to a method of using a perfusion line according to the invention, characterized in that it comprises the steps consisting in:
connecting a first flushing receptacle containing a flushing product, such as a physiological serum or glucose, to the luer type connector means of one of the fluid inlet ways of the tap comprising deformable or movable stopping means,
draining the tap and the extender by injecting into the latter a predetermined quantity of the flushing product contained in the first flushing receptacle,
connecting the distal end of the extender to an element forming a catheter,
injecting a medical treatment liquid up to the element forming a catheter with the aid of a medical treatment liquid administration receptacle connected to the luer type connector means of one of the fluid inlet ways of the tap,
flushing the extender with the aid of a flushing product so as to discharge the medical treatment liquid contained in the extender to the element forming a catheter.

Flushing the extender with a flushing product after the step for injecting the treatment liquid makes it possible to ensure a discharge of substantially all of the medical treatment liquid contained in the extender, and therefore preventing on the one hand a waste of product and on the other hand a splashing of treatment liquid on the patient or the hospital personnel during the disconnection of the catheter extender.

In addition, since substantially all the medical treatment liquid contained in the extender is discharged from the latter, the discarded waste is much less harmful for the environment.

Consequently, the method of use according to the invention makes it possible to protect the environment and to limit the risks of contact with the treatment liquids for the hospital personnel and the patient to be treated.

Preferably, the method comprises a step of filling the administration receptacle produced before the step of injecting the medical treatment liquid and consisting in:
connecting the empty administration receptacle to the luer type connector means of one of the fluid inlet ways of the tap,
connecting a preparation receptacle containing the medical treatment liquid to one of the inlet ways of the tap comprising deformable or movable stopping means,
injecting into the administration receptacle the medical treatment liquid contained in the preparation receptacle,
disconnecting the preparation receptacle.

The presence of the deformable and movable stopping means on the inlet way of the tap that receives the preparation receptacle makes it possible to prevent any splashing or running of treatment liquid during the disconnection of the preparation receptacle.

Therefore, the method of use according to the invention protects the hospital personnel and the patient to be treated from any contact with the treatment liquid during the filling of the administration receptacle.

Advantageously, the step of filling the administration receptacle comprises the steps consisting in:
connecting a second flushing receptacle containing a flushing product, such as physiological serum or glucose, to the luer type connector means of one of the inlet ways of the tap comprising deformable or movable stopping means,
injecting into the administration receptacle a predetermined quantity of flushing product contained in the second flushing receptacle so as to dilute the medical treatment liquid contained in the administration receptacle.

The use of a tap having two fluid inlet ways makes it possible to produce the mixture of the various products constituting the medical treatment liquid in a closed circuit and therefore to protect the hospital personnel and the patient to be treated from any contact with the medical treatment liquid.

Preferably, the step of filling the administration receptacle comprises a step consisting in disconnecting the second flushing receptacle after the injection of the predetermined quantity of flushing product into the administration receptacle.

Advantageously, the method comprises a step consisting in disconnecting the first flushing receptacle after the draining of the tap and of the extender.

According to one mode of applying the method, the step of flushing the extender consists in connecting a third flushing receptacle containing a flushing product, such as physiological serum or glucose, to the luer type connector means of one of the fluid inlet ways of the tap comprising deformable or movable stopping means, and in injecting into the extender a predetermined quantity of the flushing product contained in the third flushing receptacle.

According to another mode of applying the method, the step of flushing the extender consists in injecting flushing product into the extender directly from the first flushing receptacle used during the step of draining the tap and the extender.

Preferably, each flushing receptacle is a flushing syringe. Advantageously, the administration receptacle is an administration syringe, and the preparation receptacle is a preparation syringe.

However, according to one embodiment of the method, at least one or all of the receptacles could be bags directly connected to the luer type connector means of the fluid inlet ways of the tap or connected to the latter by means of extension lines having complementary luer type connector means.

Preferably, the medical treatment liquid is a chemotherapy product.

### BRIEF DESCRIPTION OF THE FIGURES

In any case, the invention will be fully understood with the aid of the following description with reference to the appended schematic drawing representing, as a non-limiting example, a form of execution of this perfusion line.

FIG. 1 is a front schematic view of a perfusion line according to the invention.

FIGS. 2 to 10 are schematic views showing various steps of the method for using the perfusion line of FIG. 1.

FIG. 11 is a schematic view showing various steps of the method for using the perfusion line of FIG. 1 according to an alternative application.

### DETAILED DESCRIPTION OF THE INVENTION

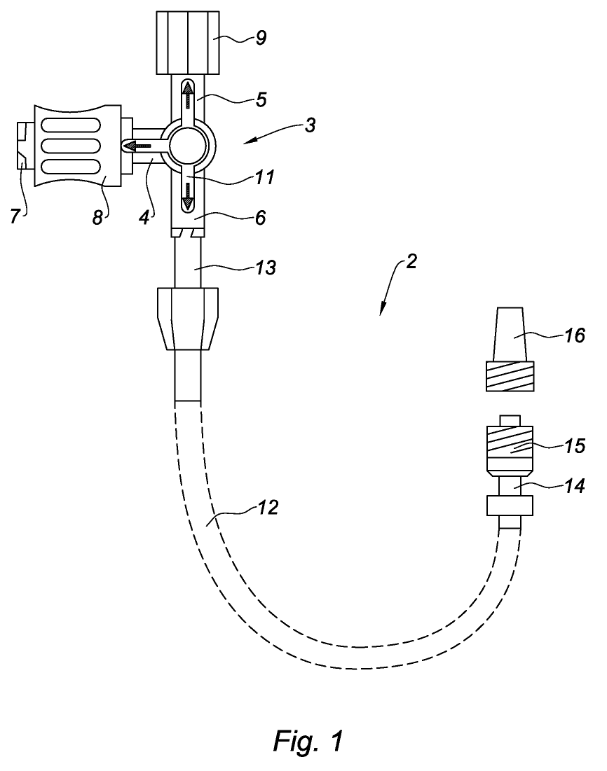

FIG. 1 represents a perfusion line 2 for the administration of chemotherapy products.

The perfusion line 2 comprises a three-way tap 3 comprising a first fluid inlet way 4 and a second fluid inlet way 5 and a fluid outlet way 6.

Each fluid inlet way 4, 5 is fitted with means for connection to a syringe. The means for connection to a syringe of each fluid inlet way comprise a fitting 7 of the female luer-lock type.

The first fluid inlet way 4 comprises stopping means that can be deformed between a first position in which the stopping means prevent a flow of fluid through the first fluid inlet way, and a second position in which the stopping means allow a flow of fluid through the first fluid inlet way. The stopping means are arranged in order to be deformed into the second position during the connection of a needleless syringe to the first fluid inlet way.

The stopping means preferably comprise an elastically deformable split stopper (not shown in the figures) housed in a rigid tubular fitting 8 and mounted on the first fluid inlet way. The edges delimiting the split of the elastically deformable stopper are arranged so as to separate elastically from one another during the connection of a needleless syringe to the first fluid inlet way so as to allow a flow of fluid through the split and therefore through the first fluid inlet way, and so as to elastically return towards one another during the disconnection of the needleless syringe from the first fluid inlet way so as to prevent a flow of fluid through the split and therefore through the first fluid inlet way. The elastically deformable split stopper consequently forms a self-stoppable inlet valve.

The second fluid inlet way 5 comprises a stopping plug 9 arranged to interact with the luer-lock type fitting of the latter.

The tap 3 comprises a handle 11 that can be rotated between a first position allowing a flow of fluid between the first fluid inlet way 4 and the second fluid inlet way 5, a second position allowing a flow of fluid between the first fluid inlet way 4 and the fluid outlet way 6, and a third position allowing a flow of fluid from the second fluid inlet way 5 and the fluid outlet way 6.

The perfusion line 2 also comprises an extender 12 whose proximal end 13 is mounted on the fluid outlet way 6 of the tap and whose distal end 14 is fitted with means of connection to a catheter. The means of connection to a catheter comprise a fitting 15 of the male luer-lock type. The extender 12 comprises a stopping plug 16 arranged to interact with the fitting 15 of the male luer-lock type.

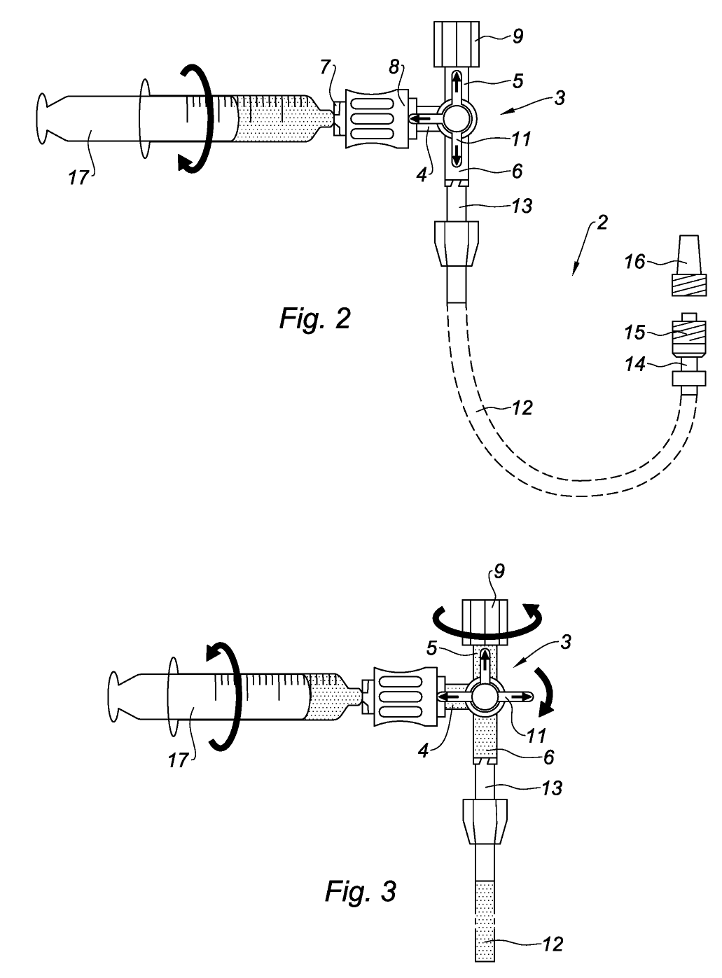

The method for using the perfusion line 2 will now be described with reference to FIGS. 2 to 10.

The method of use comprises a first step of draining the tap 3 and the extender 12 consisting in:
connecting a first flushing syringe 17 containing a flushing product, such as physiological serum or glucose, to the first fluid inlet way 4 of the tap 3, as is shown in FIG. 2,
positioning the handle 11 of the tap 3 so as to allow a flow of fluid from the first fluid inlet way 4 to the fluid outlet way 6,
draining the extender 12 up to its distal end 14 by injecting into the latter a predetermined quantity of the flushing product contained in the first flushing syringe 17,
slightly unscrewing the stopping plug 9 of the second fluid inlet way 5,
positioning the handle 11 of the tap 3 so as to allow a flow of fluid from the first fluid inlet way 4 to the second fluid inlet way 5, as is shown in FIG. 3,
draining the second fluid inlet way 5 by injecting into the latter a predetermined quantity of the flushing product contained in the first flushing syringe 17,
disconnecting the first flushing syringe 17.

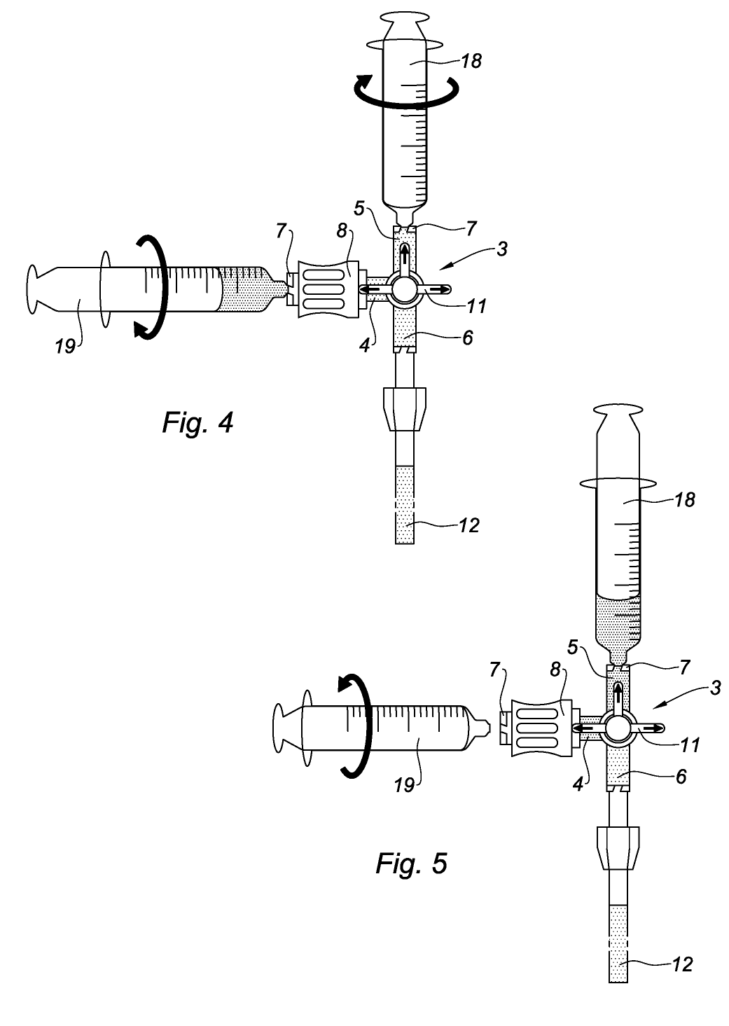

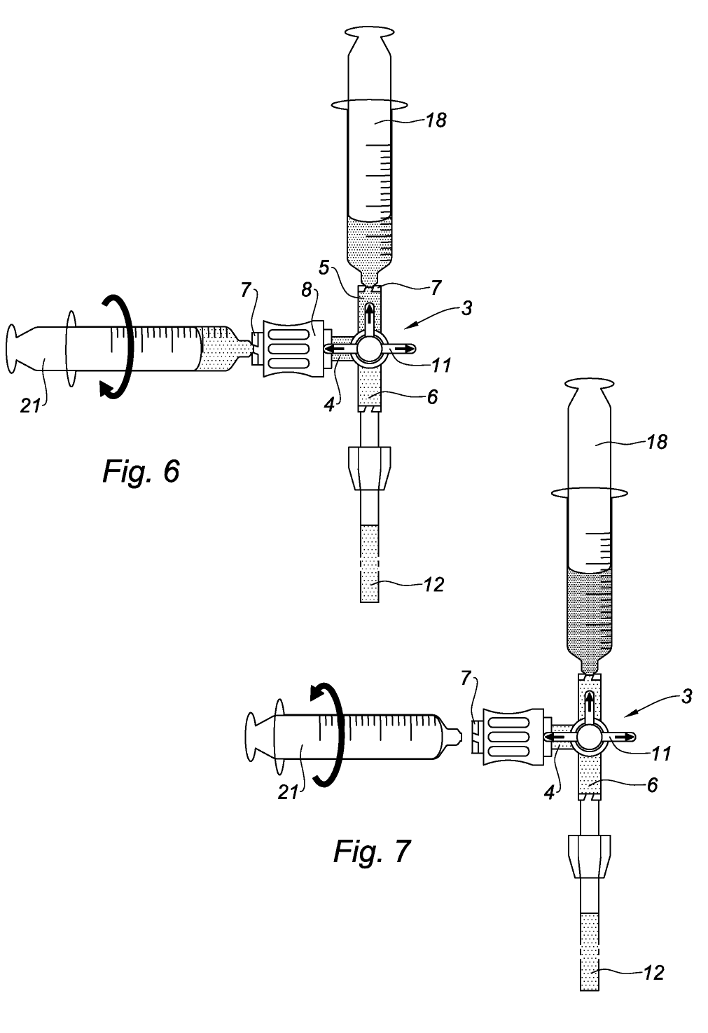

The method of use comprises a second step of filling an administration syringe consisting in:
removing the stopping plug 9 from the second fluid inlet way 5,
connecting an empty administration syringe 18 to the second fluid inlet way 5 of the tap 3, as is shown in FIG. 4,
connecting a preparation syringe 19 containing a chemotherapy product to the first fluid inlet way 4 of the tap 3 as is shown in FIG. 4,
positioning the handle 11 of the tap 3 so as to allow a flow of fluid from the first fluid inlet way 4 to the second fluid inlet way 5,
injecting into the administration syringe 18 the chemotherapy product contained in the preparation syringe 19,
disconnecting the preparation syringe 19, as is shown in FIG. 5,
connecting a second flushing syringe 21 containing the flushing product, such as physiological serum or glucose, to the first inlet way 4 of the tap 3, as is shown in FIG. 6,
injecting into the administration syringe 18 a predetermined quantity of the flushing product contained in the second flushing syringe 21 so as to dilute the chemotherapy product contained in the administration syringe 18,
disconnecting the second flushing syringe 21 after the injection of the predetermined quantity of flushing product into the administration syringe 18, as is shown in FIG. 7.

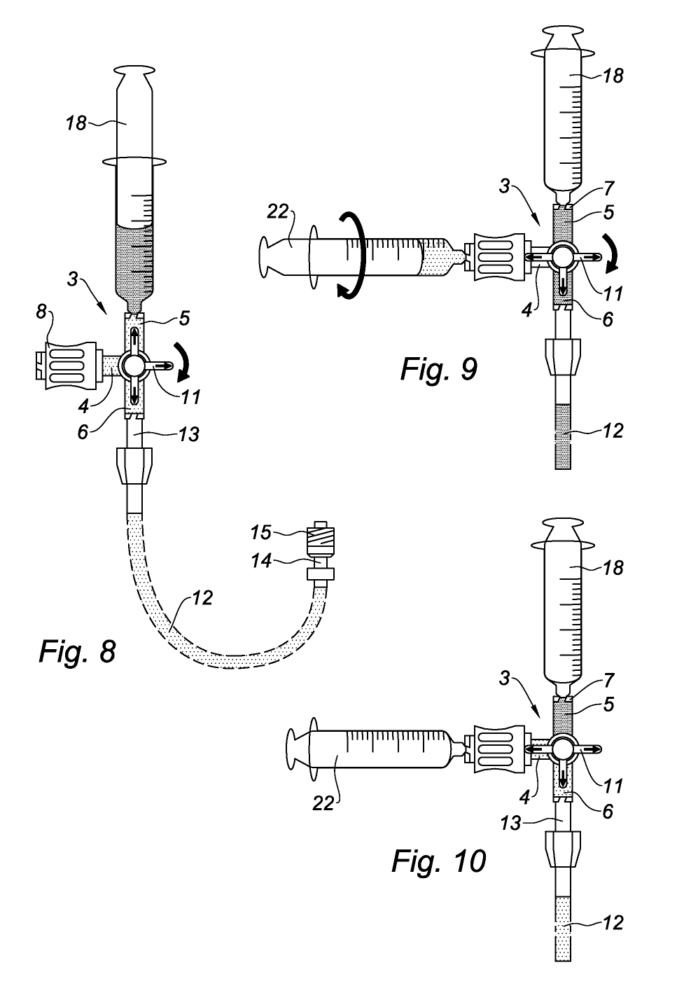

The method of use comprises a third step of administering the chemotherapy product consisting in:
connecting the distal end 14 of the extender 12 to a catheter positioned on a patient to be treated,
positioning the handle 11 of the tap 3 so as to allow a flow of fluid from the second fluid inlet way 5 to the fluid outlet way 6 of the tap 3, as is shown in FIG. 8,
administering the chemotherapy product contained in the administration syringe 18 to the patient via the extender 12 and the catheter.

The method of use finally comprises a fourth step of flushing the extender 12 consisting in:
connecting a third flushing syringe 22 containing a flushing product, such as physiological serum or glucose, to the first fluid inlet way 4 of the tap 3, as is shown in FIG. 9,
positioning the handle 11 on the tap 3 so as to allow a flow of fluid from the first fluid inlet way 4 to the fluid outlet way 6, as is shown in FIG. 9,
flushing the extender 12 by injecting into the latter a predetermined quantity of the flushing product contained in the third flushing syringe 22 so as to discharge the whole of the chemotherapy product contained in the extender 12 to the patient via the catheter, as is shown in FIG. 10.

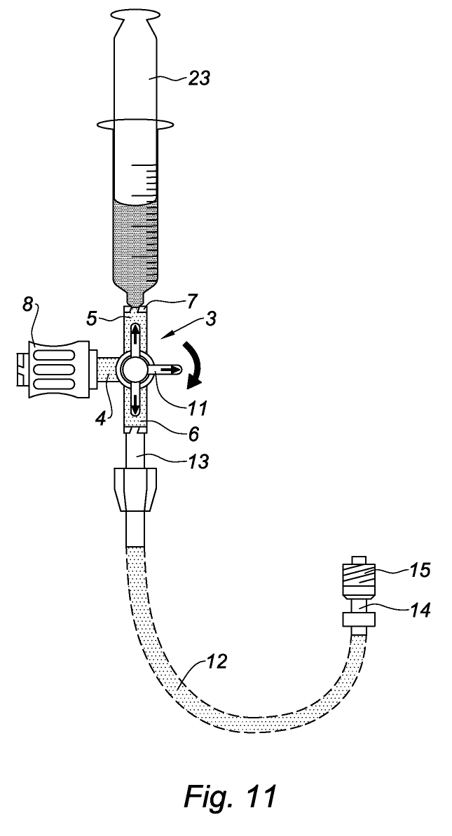

According to an alternative application of the method of use, the second step of filling and the third step of administration are replaced by a step of administration of a chemotherapy product consisting in:
removing the stopping plug 9 from the second fluid inlet way 5,
connecting an administration syringe 23 containing a chemotherapy product to the second fluid inlet way 5 of the tap 3, as is shown in FIG. 11,
positioning the handle 11 of the tap 3 so as to allow a flow of fluid from the second fluid inlet way 5 to the fluid outlet way 6, as is shown in FIG. 11,
administering the chemotherapy product contained in the administration syringe 23 to the patient via the extender 12 and the catheter.

According to another alternative of the method of use, the step of flushing the extender consists in injecting the flushing product into the extender 12 directly from the first flushing syringe 17 used during the step of draining the tap 3 and the extender 12.

It goes without saying that the invention is not limited solely to the form of execution of this perfusion line described above as an example; on the contrary, it covers all the variant embodiments.

What is claimed is:

1. Perfusion line for administration of medical treatment liquids, such as chemotherapy products, to a patient, comprising:
a tap comprising at least two fluid inlet ways and a fluid outlet way, each fluid inlet way being fitted with luer type connector means arranged to allow the connection of a receptacle, such as a syringe, at least one of the fluid inlet ways comprising stopping means that can be deformed or moved between a first position in which the stopping means prevent a flow of fluid through the fluid inlet way comprising the said stopping means, and a second position in which the stopping means allow a flow of fluid through the fluid inlet way comprising the said stopping means, the stopping means being arranged to be deformed or moved into the second position when a receptacle, such as a syringe, is connected to the fluid inlet way comprising the said stopping means,
an extender of which one of the ends, called the proximal end, is mounted on the fluid outlet way of the tap and of which the other end, called the distal end, is fitted with means for connection to an element forming a catheter.

2. Perfusion line according to claim 1, wherein the stopping means comprise an elastically deformable split stopper.

3. Perfusion line according to claim 1, wherein the means of connection to an element forming a catheter comprise a fitting of the male luer-lock type.

4. Perfusion line according to claim 1, wherein the luer type connector means of each fluid inlet way comprise a fitting of the female luer-lock type.

5. Perfusion line according to claim 1, wherein the tap is a three-way tap.

6. Method of using the perfusion line of claim 1, the method comprising:
connecting a first flushing receptacle containing a flushing product, such as a physiological serum or glucose, to the luer type connector means of one of the fluid inlet ways of the tap comprising deformable or movable stopping means,
draining the tap and the extender by injecting into the latter a predetermined quantity of the flushing product contained in the first flushing receptacle,
connecting the distal end of the extender to an element forming a catheter,
injecting a medical treatment liquid up to the element forming a catheter with the aid of a medical treatment liquid administration receptacle connected to the luer type connector means of one of the fluid inlet ways of the tap,
flushing the extender with the aid of a flushing product so as to discharge the medical treatment liquid contained in the extender to the element forming a catheter.

7. Method according to claim 6, further comprising filling the administration receptacle produced before said injecting the medical treatment liquid and further comprising:
connecting the empty administration receptacle to the luer type connector means of one of the fluid inlet ways of the tap,
connecting a preparation receptacle containing the medical treatment liquid to one of the inlet ways of the tap comprising deformable or movable stopping means,
injecting into the administration receptacle the medical treatment liquid contained in the preparation receptacle,
disconnecting the preparation receptacle.

8. Method according to claim 7, wherein said filling the administration receptacle comprises:
connecting a second flushing receptacle containing a flushing product, such as physiological serum or glucose, to the luer type connector means of one of the inlet ways of the tap comprising deformable or movable stopping means,
injecting into the administration receptacle a predetermined quantity of flushing product contained in the second flushing receptacle so as to dilute the medical treatment liquid contained in the administration receptacle.

9. Method according to claim 8, wherein said filling the administration receptacle comprises disconnecting the second flushing receptacle after the injection of the predetermined quantity of flushing product into the administration receptacle.

10. Method according to claim 6, further comprising disconnecting the first flushing receptacle after the draining of the tap and of the extender.

11. Method according to claim 6, wherein said flushing the extender comprises connecting a third flushing receptacle containing a flushing product, such as physiological serum or glucose, to the luer type connector means of one of the fluid inlet ways of the tap comprising deformable or movable stopping means, and in injecting into the extender a predetermined quantity of the flushing product contained in the third flushing receptacle.

12. Method according to claim 6, wherein said flushing the extender comprises injecting flushing product into the extender directly from the first flushing receptacle used during said draining the tap and the extender.

13. Method according to claim 6, wherein each flushing receptacle is a flushing syringe.

14. Method according to claim 6, wherein the administration receptacle is an administration syringe.

15. Method according to claim 7, wherein the preparation receptacle is a preparation syringe.

16. Method according to claim 6, wherein the medical treatment liquid is a chemotherapy product.
